(19) United States
(12) Patent Application Publication
(10) Pub. No.: US 2005/0215471 A1
(43) Pub. Date: Sep. 29, 2005

(54) Compositions of bacteria and alpha-lactalbumin and uses thereof

(76) Inventor: Hemant K. Sabharwal, Irvington, NY (US)

Correspondence Address: KLAUBER & JACKSON, 411 HACKENSACK AVENUE, HACKENSACK, NJ, 07601, US

(21) Appl. No.: 10/972,747

(22) Filed: Oct. 25, 2004

Related U.S. Application Data
(63) Continuation of application No. 10/123,875, filed on Apr. 16, 2002, now abandoned.
(60) Provisional application No. 60/283,901, filed on Apr. 16, 2001.
(60) Provisional application No. 60/292,436, filed on May 22, 2001.

(51) Int. Cl. A61K 38/17; C12N 1/20

(52) U.S. Cl. 514/12; 435/69.1; 435/252.9

(57) ABSTRACT

The present invention relates to nutriceutical compositions comprising bifidobacteria, optionally with a lactic acid-fermentation bacterial strain, and monomeric alpha-lactalbumin. The compositions are useful as food additives and have surprising levels of anti-bacterial activity. The present invention is also related to methods for treating and/or preventing gastric ulcers comprising administering a composition of the present invention.

The present application claims priority benefits of U.S. Provisional Application No. 60/283,901 filed Apr. 16, 2001 and U.S. Provisional Application No. 60/292,436 filed May 22, 2001, the disclosures of which are incorporated herein by reference in their entirety.

### 1. FIELD OF THE INVENTION

The present invention relates to compositions comprising bifidobacteria, optionally with a lactic acid-fermentation bacterial strain, and monomeric alpha-lactalbumin. The compositions are useful as food additives and have surprising levels of anti-bacterial activity. The present invention is also related to methods for treating and/or preventing gastric ulcers comprising administering a composition of the present invention.

### 2. BACKGROUND OF THE INVENTION

Probiotics are a class of microorganisms that are defined as live microbial organisms that beneficially affect the animal and human hosts. The beneficial effects include improvement of the microbial balance of the intestinal microflora or by improving the properties of the indigenous microflora. A better understanding of probiotics in man and animals can be found in the following publications. Fuller R: Probiotics in Man and Animals, J. Appl. Bacteriol 1989;66:365-365-378 and Havenaar R, Brink B, Huis In't Veld JHJ: Selection of Strains for Probiotic Use. In Scientific Basis of the Probiotic Use, ed. R. Fuller, Chapman and Hall, London UK, 1992. The known benefits of enteral administration of probiotic microorganisms include enhanced host defense to disease; improving colonization resistance of the harmful microflora and numerous other areas of health promotion. Probiotics have been suggested to play an important role in the formation or establishment of a well-balanced, indigenous, intestinal microflora in newborn children or adults receiving high doses of antibiotics.

Bifidobacterium infantis is a Gram-positive, strictly anaerobic, fermentative rod, and has been recommended for use as probiotics. Bifidobacterium infantis is the predominant form of Bifidobacterium in breast fed infant feces.

Lactic acid bacteria and specific strains of Lactobacillus have also been widely recommended for use as probiotics. See, for example, Gilliland SE: Health and Nutritional Benefits from Lactic Acid Bacteria. Micro Rev. 1990;87;175-188 and Gorbach SL: Lactic Acid Bacteria and Human Health. Annals of Med. 1990;22-37-41. One of the more recently studied probiotics is Lactobacillus reuteri. This ubiquitous microorganism resides in the gastrointestinal tract of humans and animals and produces a potent, broad spectrum antimicrobial substance called reuterin. The inhibition of growth of Escherichia, Salmonella, Shigella, Listeria, Campylobacter, Clostridium and species of Staphylococcus by reuterin has been reported. See for example, Axeisson L T, et al (1989), Production of a Broad Spectrum Antimicrobial Substance by Lactobacillus reuteri, Microbial Ecology in Health and Disease 2, 131-136.

Of the intestinal lactic acid bacteria (LAB), L. reuteri is considered a major species. L. reuteri is a dominant heterofermentative Lactobacillus species residing in the gastrointestinal tract of healthy humans and most animals. Like other lactobacilli, L. reuteri produces acidic metabolic end-products which have considerable antimicrobial activity. It has been recently discovered that metabolism of glycerol by L. reuteri can result in excretion of a metabolic intermediate, 3-hydroxpropionaldehyde, or reuterin. See Axelsson, “Production of a Broad Spectrum Antimicrobial Substance by Lactobacillus reuteri,” Microbial Ecology in Health and Disease, 2:131-136, 1989. Reuterin has been shown to have antimicrobial activity against a variety of organisms including Gram-positive and Gram-negative bacteria, yeast, molds and protozoa. See Chung, et al., “In Vitro Studies on Reuterin Synthesis by Lactobacillus reuteri,” Microbial Ecology in Health and Disease, 2:137-144, 1989. It is suspected that the antimicrobial activity of reuterin contributes to the survival of L. reuteri within the gastrointestinal ecosystem. Likewise, L. acidophilus is a normal inhabitant of the human gastrointestinal tract and is a Gram-positive rod widely used in the dairy industry. L. acidophilus is a homofermentative species, fermenting mainly hexose sugar, yielding predominantly lactic acid (85-95%): The use of L. acidophilus predates the 20th century.

Multimeric, biologically active alpha-lactalbumin has also been shown to provide anti-bacterial effects. Hakansson et al., 2000, Molecular Microbiology 35:589-600.

### 3. SUMMARY OF THE INVENTION

The present invention is directed to a composition comprising an admixture of the following ingredients: bifidobacteria, and substantially purified monomeric alpha-lactalbumin. Once the ingredients are mixed together, the acid produced by the bacteria converts the monomeric alpha-lactalbumin into multimeric alpha-lactalbumin. In a preferred embodiment, the bifidobacteria are present at a concentration of at least 10⁶ colony forming units (cfu) per ml and the alpha-lactalbumin is present at a concentration of at least 0.1 mg/ml. In an optional embodiment, the composition further comprises the ingredient of a lactic acid bacterial strain, and the total concentration of bacteria is at least 10⁶ cfu/ml. In another optional embodiment, the composition further comprises the ingredient of an acid producing fungal organism. The compositions of the present invention can be added to a variety of dairy or non-dairy food stuffs including, but not limited to, infant formula; baby food; fruit drinks; milk products like ice cream, milk, milk shakes and yogurt; sports and nutrition drinks; processed cheese, salad dressing and soup, in order to enhance the nutritional value of the food stuffs. As used herein, non-dairy food stuffs refers to food stuffs not containing or not derived from milk. In another embodiment, the bacterial ingredient is in an inert form, i.e., a form in which the bacteria are not growing or utilizing metabolites or producing acid. Such inert forms include, but are not limited to, a lyophilized form or a dry powered form.

The present invention is also directed to a method for treating and/or preventing a gastric ulcer caused by a bacterial infection comprising administering to a patient in need thereof, a composition comprising an admixture of the following ingredients: bifidobacteria and substantially purified monomeric alpha-lactalbumin. In a preferred embodiment, the bifidobacteria are present at a concentration of at least 10⁶ colony forming units (cfu) per ml and the alpha-lactalbumin is present at a concentration of at least 0.1 mg/ml. In an optional embodiment, the composition further comprises the ingredient of a lactic acid bacterial strain, and the total concentration of bacteria is at least 10⁶ cfu/ml. In another optional embodiment, the composition further comprises the ingredient of an acid producing fungal organism.

The compositions of the present invention provide a surprising synergistic (i.e., more than additive) anti-bacterial effect against an unexpectedly broad spectrum of microorganisms.

The present invention is also directed to a method of producing a composition comprising mixing bifidobacteria with substantially purified monomeric alpha-lactalbumin. In another embodiment, the method comprises mixing bifidobacteria, a lactic acid bacterial strain and substantially purified monomeric alpha-lactalbumin. In alternate embodiments, the bacteria are added in an inert form.

The present invention is also directed to a dairy or non-dairy food stuff comprising bifidobacteria and substantially purified monomeric alpha-lactalbumin. In another embodiment, the food stuff comprises bifidobacteria, a lactic acid bacterial strain and substantially purified monomeric alpha-lactalbumin.

The present invention is also directed to a method of making multimeric alpha-lactalbumin comprising culturing an acid producing bacterial strain with monomeric alpha-lactalbumin for a time sufficient to produce multimeric alpha-lactalbumin.

### 4. DETAILED DESCRIPTION OF THE INVENTION

The present invention is directed to a composition comprising an admixture of the following ingredients: bifidobacteria, and substantially purified monomeric alpha-lactalbumin. Once the ingredients are mixed together, the acid produced by the bacteria converts the monomeric alpha-lactalbumin into multimeric alpha-lactalbumin. In a preferred embodiment, the bifidobacteria are present at a concentration of at least 10⁶ colony forming units (cfu) per ml and the alpha-lactalbumin is present at a concentration of at least 0.1 mg/ml. In an optional embodiment, the composition further comprises the ingredient of a lactic acid bacterial strain, and the total concentration of bacteria is at least 10⁶ cfu/ml. In another optional embodiment, the composition further comprises the ingredient of an acid producing fungal organism. The compositions of the present invention can be added to a variety of dairy or non-dairy food stuffs including, but not limited to, infant formula; baby food; fruit drinks; milk products like ice cream, milk, milk shakes and yogurt; sports and nutrition drinks; processed cheese, salad dressing and soup, in order to enhance the nutritional value of the food stuffs. As used herein, non-dairy food stuffs refers to food stuffs not containing or not derived from milk. In another embodiment, the bacterial ingredient is in an inert form, i.e., a form in which the bacteria is not growing or utilizing metabolites or producing acid. Such inert forms include, but are not limited to, a lyophilized form or a dry powered form.

The present invention is also directed to a method for treating and/or preventing a gastric ulcer caused by bacterial infection comprising administering to a patient in need thereof, a composition comprising an admixture of the following ingredients: bifidobacteria and substantially purified monomeric alpha-lactalbumin. In a preferred embodiment, the bifidobacteria are present at a concentration of at least 10⁶ colony forming units (cfu) per ml and the alpha-lactalbumin is present at a concentration of at least 0.1 mg/ml. In an optional embodiment, the composition further comprises the ingredient of a lactic acid bacterial strain, and the total concentration of bacteria is at least 10⁶ cfu/ml. In another optional embodiment, the composition further comprises the ingredient of an acid producing fungal organism.

The present invention is also directed to a method of producing a composition comprising mixing bifidobacteria with substantially purified monomeric alpha-lactalbumin. In another embodiment, the method comprises mixing bifidobacteria, a lactic acid bacterial strain and substantially purified monomeric alpha-lactalbumin.

The present invention is also directed to a dairy or non-dairy food stuff comprising bifidobacteria and substantially purified monomeric alpha-lactalbumin. In another embodiment, the food stuff comprises bifidobacteria, a lactic acid bacterial strain and substantially purified monomeric alpha-lactalbumin.

The present invention is also directed to a method of making multimeric alpha-lactalbumin comprising culturing an acid producing bacterial strain with monomeric alpha-lactalbumin for a time sufficient to produce multimeric alpha-lactalbumin.

4.1 Microorganisms

The microorganisms useful in the present invention are bifidobacteria and lactic acid bacteria. Illustrative strains of bifidobacteria include, but are not limited to, Bifidobacterium infantis, Bifidobacterium bifidum, Bifidobacterium breve, Bifidobacterium longum, and Bifidobacterium adolescentis.

Lactic acid bacterial strains useful in the present invention include, but are not limited to, Lactobacilli strains, e.g., Lactobacillus reuteri, Lactobacillus fermenyum (fermetum), Lactobacillus acidophillus, Thermobacterium yoghourtii, Lactobacillus bulgaricus, Streptococcus cremaris, Streptococcus paracitrooarus, Streptococcus thermophillus, Streptococcus lactis (especially the Taette strain, N group), Lactobacillus casei, and Streptococcus kefir. Optionally, fungal organisms that produce lactic acid are also useful; such organisms include, but are not limited to, Saccharomyces fragilis.

Cultures of the microorganisms are commercially available and are usually supplied in an inert form, for example, lyophilized, or in a powdered form. For example, the bacterial strains can be obtained from the American Type Culture Collection, Manassas, Va., American Health, Inc., and Morinaga Milk Industry. Once obtained, the cultures can be grown (fermented) and isolated according to any standard procedure in the art and lyophilized in a manner known to those of skill in the art to keep the bacteria viable. Optionally, before lyophilization, a carrier/cryoprotectant can be added. Illustrative examples of a cryoprotectant include, but are not limited to, whey protein concentrate, lactose, maltrodextrin, N-[tris(hydroxymethyl)methyl]glycine (tricine), trehalose, maltose, sucrose, cellobiose, glucose, galactose, fructose, inositol, sorbitol, and glycerol.

4.2 Alpha-Lactalbumin

Substantially purified monomeric alpha-lactalbumin can be obtained from a number of sources, including mammalian milk and milk products, and from commercial sources, such as Sigma Chemical Co., St. Louis, Mo. and Davisco Foods, Eden Prairie, Minn. The alpha-lactalbumin can be in a dry or liquid (wet) form. In one embodiment of the present invention, the substantially purified monomeric alpha-lactalbumin before being added to the bacteria is at least 10% pure. In another embodiment, the monomeric alpha-lactalbumin is at least 15%, 20%, 25%, 30%, 35%, 40%, 45% or 50% pure. In yet another embodiment, the substantially purified monomeric alpha-lactalbumin before being added to the bacteria is 55%, 60%, 65%, 70%. 75%, 80%, 85%, 90% or 95% pure. It is understood that the percentage is any percentage recited within the range. In a preferred embodiment, the monomeric alpha-lactalbumin is derived from bovine milk.

4.3 Methods of Preventing/Treating Ulcers

The present invention is also directed to a method for treating and/or preventing a gastric ulcer caused by a bacterial infection. In one embodiment, the method comprises administering to a patient in need thereof, a composition comprising an admixture of the following ingredients: bifidobacteria and substantially purified monomeric alpha-lactalbumin, in an amount sufficient to treat or prevent a gastric ulcer. In another embodiment, the composition further comprises the ingredient of a lactic acid bacterial strain. In yet another embodiment, the composition further comprises the ingredient of an acid producing fungal organism. As used herein, treating or preventing a gastric ulcer includes ameliorating the symptoms of the gastric ulcer.

The amount of the pharmaceutical composition of the invention which will be effective in the treatment or prevention of a particular disorder or condition will depend on the nature of the disorder or condition, and can be determined by standard clinical techniques. In addition, in vitro assays may optionally be employed to help identify optimal dosage ranges. The precise dose to be employed in the formulation will also depend on the route of administration, and the seriousness of the disease or disorder, and should be decided according to the judgment of the practitioner and each patient's circumstances. However, suitable dosage ranges for the bifidobacteria or bifidobacteria and lactic acid bacterial strain are generally from about 1.0 cfu/kg to about 1×10¹⁰ cfu/kg; optionally from about 1.0 cfu/kg to about 1×10⁸ cfu/kg; optionally from about 1×10² cfu/kg to about 1×10⁸ cfu/kg; optionally from about 1×10⁴ cfu/kg to about 1×10⁸ cfu/kg. Effective doses may be extrapolated from dose-response curves derived from in vitro or animal model test systems. Suitable dosage ranges for monomeric alpha-lactalbumin are generally from about 0.001 mg/kg to about 1 mg/kg; optionally from about 0.01 mg/kg to about 1 mg/kg; optionally from about 0.1 mg/kg to about 0.5 mg/kg.

The compositions of the present invention can be formulated with a pharmaceutically acceptable carrier for administration to a subject. In a preferred embodiment, the subject is a human subject. In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water, saline solutions and aqueous dextrose and glycerol solutions can also be employed as liquid carriers. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin. Such compositions will contain a therapeutically effective amount of the composition of bacteria and alpha-lactalbumin together with a suitable amount of carrier so as to provide the form for proper administration to the patient. The formulation should suit the mode of administration.

The present invention also provides a pharmaceutical pack or kit comprising one or more containers filled with one or more of the ingredients of the pharmaceutical compositions of the invention. For example, the kit can comprise a vial containing a pharmaceutical composition comprising bifidobacteria, preferably in lyophilized form, and alpha-lactalbumin. In another example, the kit can comprise two vials, one containing a pharmaceutical composition comprising bifidobacteria, preferably in lyophilized form, and the other vial containing a pharmaceutical composition comprising alpha-lactalbumin. Optionally associated with such container(s) can be instructions for use of the kit and/or a notice in the form prescribed by a governmental agency regulating the manufacture, use or sale of pharmaceuticals or biological products, which notice reflects approval by the agency of manufacture, use or sale for human administration.

### 5. EXAMPLE

### 5.1 Bacterial Cultures

All bacterial strains are stored lyophilized at −80° C. The cultures of Lactobacillus acidophilus, Lactobacillus bifidus and Bifidobacterium longum are thawed and transferred to blood agar plates and grown overnight at 37° C. Group A Streptococci is grown overnight at 37° C. in Todd-Hewitt Broth (THB), until early log phase with an O.D. of 0.70 at 600 μm. At this absorbance, the concentration is approximately 10′ cfu/ml.

Bacterial suspensions (log phase cultures, 0.1 ml, 10⁶-10⁸ cfu/ml) are made in THB (0.9 ml) or incubated with the composition to be tested. The mixtures are incubated at 37° C., and 0.1 ml samples are withdrawn at times 0, 1, 2 and 4 hours. The samples are serially diluted and the relevant dilutions are plated on blood agar plates. The p-hemolytic colonies are counted after overnight incubation at 37° C., and the concentration of viable bacteria determined.

5.2 Alpha-Lactalbumin

The different sources of alpha-lactalbumin used to prepare the compositions are either from bovine milk (Sigma Chemical Co., St. Louis, Mo. or Davisco Foods, Eden Prairie, Minn.) or from human milk (Sigma Chemical Co., St. Louis, Mo.). All preparations of alpha-lactalbumin are supplied as freeze-dried powder.

5.3 Preparation of the Compositions

The compositions are prepared as follows:
1) 50 mg of Lactobacillus acidophilus is mixed with 50 mg Lactobacillus bifidus, producing probiotic mixture 1 (10⁹ cfu total);
2) 50 mg of Lactobacillus acidophilus is mixed with 50 mg Bifidobacterium longum, producing probiotic mixture 2 (10⁹ cfu total);
3) 50 mg bovine alpha-lactalbumin from Davisco Foods is added to probiotic mixture 1 and this mixture is termed B1;
4) 50 mg bovine alpha-lactalbumin from Sigma is added to probiotic mixture 1 and this mixture is termed B2;
5) 50 mg bovine alpha-lactalbumin from Davisco Foods is added to probiotic mixture 2 and this mixture is termed B3;
6) 50 mg bovine alpha-lactalbumin from Sigma is added to probiotic mixture 2 and this mixture is termed B4;
7) 50 mg human alpha-lactalbumin from Sigma is added to probiotic mixture 1 and this mixture is termed H1;
8) 50 mg human alpha-lactalbumin from Sigma is added to probiotic mixture 2 and this mixture is termed H2;
9) 100 mg of Bifidobacterium longum (10⁹ cfu total) is added to 50 mg bovine alpha-lactalbumin from Davisco Foods and this mixture is termed L1; and
10) 100 mg of Bifidobacterium longum (10⁹ cfu total) is added to 50 mg bovine alpha-lactalbumin from Sigma and this mixture is termed L2.

Each of mixtures B1, B2, B3, B4, H1, H2, L1 and L2 are divided into 3 equal portions of 50 mg each.

5.4 Bactericidal Activity of the Mixtures

The bactericidal activity of the mixtures is tested as follows:

1) 10 ml of sterile physiological saline is added to one 50 mg portion of the mixtures. 10 mg of alpha-lactalbumin is dissolved in 10 ml saline as a control.

2) The solutions are incubated for 24 hours at 37° C.

3) At times 0, 2, 4, 6, and 24 hours, 0.5 ml of each solution is withdrawn, centrifuged for 15 minutes and the supernatant is tested for anti-bacterial activity, as described in Section 5.1.

4) To the second 50 ml portion, 10 ml sterile saline is added along with 10⁷ cfu of Group A Streptococci. 10⁷ cfu of Group A Streptococci is also added to alpha-lactalbumin alone as a control.

5) The solutions are incubated at 37° C. for 24 hours.

6) At times 0, 2, 6, and 24 hours, 0.5 ml of each solution is withdrawn, diluted to 10⁻⁵, and 20 μl of each dilution is plated on blood agar plates.

7) The plates are incubated overnight at 37° C., and the β-hemolytic colonies are counted.

The present invention is not to be limited in scope by the specific embodiments described herein. Indeed, various modifications of the invention in addition to those described herein will become apparent to those skilled in the art from the foregoing description and accompanying figures. Such modifications are intended to fall within the scope of the appended claims.

Various publications are cited herein, the disclosures of which are incorporated by reference in their entireties.

1-25. (canceled)

26: A method for producing multimeric alpha-lactalbumin comprising culturing an acid producing microorganism with substantially purified monomeric alpha-lactalbumin for a time sufficient to produce multimeric alpha-lactalbumin.

27: The method of claim 26 wherein the alpha-lactalbumin is present at a concentration of at least about 0.1 mg/ml.

28: The method of claim 26 wherein the acid producing microorganism is bifidobacteria.

29: The method of claim 26 wherein the acid producing microorganism is present in a concentration of at least about 10⁶ cfu/ml.

30: The method of claim 26 wherein the acid producing microorganism is lyophilized.

31: The method of claim 26 wherein the alpha-lactalbumin is mammalian.

32: A method of treating a bacterial infection in a patient comprising the steps of
(a) culturing an acid producing microorganism with substantially purified monomeric alpha-lactalbumin for a time sufficient to produce multimeric alpha-lactalbumin; and
(b) administering a therapeutically effective amount of the multimeric alpha-lactalbumin to the patient.

33: A method of treating a gastric ulcer in a patient comprising die steps of
(a) culturing an acid producing microorganism with substantially purified monomeric alpha-lactalbumin for a time sufficient to produce multimeric alpha-lactalbumin; and
(b) administering a therapeutically effective amount of the multimeric alpha-lactalbumin to the patient.
